(19) United States
(12) Patent Application Publication
(10) Pub. No.: US 2005/0009897 A1
(43) Pub. Date: Jan. 13, 2005

(54) Method of treatment

(76) Inventors: Karen Anderson, Collegeville, PA (US); Mary Ann Lukas, Philadelphia, PA (US); Terry Holcslaw, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/473,667

(22) PCT Filed: Apr. 2, 2002
(86) PCT No.: PCT/US02/10299

Related U.S. Application Data
(60) Provisional application No. 60/280,859, filed on Apr. 2, 2001.

(51) Int. Cl. A61K 31/403

(52) U.S. Cl. 514/411

(57) ABSTRACT

This invention relates to a method of maintaining glycemic control in diabetic hypertensive patients which comprises administering carvedilol to a subject in need thereof.

### FIELD OF THE INVENTION

This invention relates to a method of maintaining glycemic control in diabetic hypertensive patients which comprises administering carvedilol to a subject in need thereof.

### BACKGROUND OF THE INVENTION

Hypertension is common in diabetic patients, and the coexistence of diabetes and hypertension confers an increased risk for the development of cardiovascular and renal disease. As many as 40% of hospitalized non-insulin-dependent diabetics are hypertensive (Pacy et al., Prevalence of hypertension in white, black, and Asian diabetics in a district hospital diabetic clinic. Diabetic Medicine 1982;5:125-130). Medical management of these patients is complex as either condition and/or its pharmacological treatment has the potential to exacerbate the pathophysiology of the other. For example, the adverse renal effects of diabetes can produce hypertension while thiazide diuretics prescribed for hypertension may induce insulin resistance and consequent compensatory hyperinsulinemia (Furman, Impairment of glucose tolerance produced by diuretics and other drugs. Pharmacol Ther. 1981; 12:613-649; Lewis et al., Deterioration of glucose tolerance in hypertensive patients on prolonged diuretic treatment. Lancet 1976;1:564-566′). Selective β₁-blockers also reportedly increase insulin resistance and worsen glycemic control (Giugliano et al., Metabolic and cardiovascular effects of carvedilol, and atenolol in non-insulin-dependent diabetes mellitus and hypertension. Ann Int Med 1997; 126:955-959; Jacob et al., Differential effect of chronic treatment with two beta-blocking agents on insulin sensitivity: the carvedilol-metoprolol study. J Hypertension 1996; 14:489494; Pollare et al., Sensitivity to insulin during treatment with atenolol and metoprolol: a randomised, double-blind study of the effects on carbohydrate and lipoprotein metabolism in hypertensive patients. Br Med J 1989;298:1152-7; UK Prospective Diabetes Study Group, Tight blood pressure control and risk of macrovascular and microvascular complications in type 2 diabetes. Br Med J 317:703-713, 1998; Wright et al., Beta-adrenoceptor blocking drugs and blood sugar control in diabetes mellitus. Br Med J 1:159-161, 1979). Consequently, some physicians are reluctant to prescribe β-blockers to hypertensive diabetic patients.

Hypertension and diabetes are also recognized risk factors for development of congestive heart failure, and approximately 15-20% of patients with congestive heart failure have concurrent diabetes mellitus. Thus, despite the recent acceptance of beta-blockers as part of standard poly-therapy in heart failure, studies that report that beta-blocking agents such as metoprolol, atenolol, and propranolol significantly reduce glycemic control in hypertensive patients (Groop L, et al., Influence of beta-blocking drugs on glucose metabolism in patients with non-insulin-dependent diabetes mellitus. Acta Med Scand 1982; 211:7-12; Pollare et al., Sensitivity to insulin during treatment with atenolol and metoprolol: a randomised, double-blind study of the effects on carbohydrate and lipoprotein metabolism in hypertensive patients. Br Med J 1989;298:1152-7) potentially elicit concerns regarding the use of beta-blockers in diabetic heart failure patients.

Carvedilol is a novel, multiple action drug approved for treatment of mild-to-moderate hypertension and mild-to-moderate heart failure (Ruffolo et al., Recent observations with beta-adrenoceptor blockade—beneficial effects in hypertension and heart failure. Am J Hypertension 11(1 Part 2 Suppl S):9S-14S, 1998). Carvedilol is a non-selective β-blocker and selective α₁-receptor blocker with coincident anti-oxidant properties. In extensive clinical trials, carvedilol has been shown to be as effective as other β-blockers, angiotensin converting enzyme inhibitors or calcium channel blockers in lowering blood pressure. Importantly, carvedilol increases insulin sensitivity in both nondiabetic (Jacob et al., Differential effect of chronic treatment with two beta-blocking agents on insulin sensitivity: the carvedilol-metoprolol study. J Hypertension 1996; 14:489-494), and diabetic (Giugliano et al., Metabolic and cardiovascular effects of carvedilol, and atenolol in non-insulin-dependent diabetes mellitus and hypertension. Ann Int Med 1997; 126:955-959) hypertensive individuals. In a randomized, double-blind, controlled trial involving 45 hypertensive diabetic patients carvedilol increased insulin sensitivity and glucose disposal while it decreased HbA1c, fasting glucose, fasting insulin, and lipid peroxidation (Giugliano et al., 1997). In contrast, despite equivalent antihypertensive effect, atenolol decreased insulin sensitivity and glucose disposal and increased HbA1c, fasting glucose, and fasting insulin. It had no effect lipid peroxidation (Giugliano et al., 1997).

According to the instant invention, carvedilol represents an antihypertensive therapeutic option for treatment of hypertensive type II diabetic patients with a unique ability, relative to selective β₁-blockers, to reduce blood pressure without compromising glycemic control. The purpose of this study is to compare the effects of carvedilol to the widely-prescribed β₁-selective antagonist metoprolol on a measure of glycemic control, glycosylated hemoglobin (HbA1c), in hypertensive patients with non-insulin-dependent diabetes mellitus.

### SUMMARY OF THE INVENTION

The present invention provides a method of maintaining glycemic control in diabetic hypertensive patients which comprises administering carvedilol to a subject in need thereof.

### DETAILED DESCRIPTION OF THE INVENTION

1-(Carbazol-4-yloxy-3-[[2-(o-methoxyphenoxy)ethyl]amino]-2-propanol is known as carvedilol. This compound has the following structure: 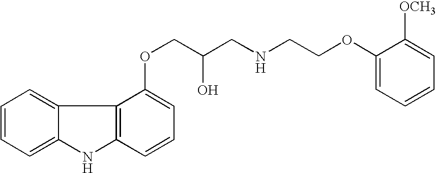
and is claimed in U.S. Pat. No. 4,503,067 (assigned to Boehringer Mannheim, GmbH, Mannheim-Waldhof, Fed. Rep. of Germany), issued Mar. 5, 1985. Reference should be made to said patent for its full disclosure, including the methods of preparing and using this compound. The entire disclosure of the '067 patent is incorporated herein by reference.

Carvedilol is a novel multiple action drug useful in the treatment of mild to moderate hypertension. Carvedilol is known to be both a competitive non-selective β-adrenoceptor antagonist and a vasodilator, and is also a calcium channel antagonist at higher concentrations. The vasodilatory actions of carvedilol result primarily from α₁-adrenoceptor blockade, whereas the β-adrenoceptor blocking activity of the drug prevents reflex tachycardia when used in the treatment of hypertension. These multiple actions of carvedilol are responsible for the antihypertensive efficacy of the drug in animals, particularly in humans. See Willette, R. N., Sauermelch, C. F. & Ruffolo, R. R., Jr. (1990) Eur. J. Pharmacol., 176, 237-240; Nichols, A. J., Gellai, M. & Ruffolo, R. R., Jr. (1991) Fundam. Clin. Pharmacol., 5, 25-38; Ruffolo, R. R., Jr., Gellai, M., Hieble, J. P., Willette, R. N. & Nichols, A. J. (1990) Eur. J. Clin. Pharmacol., 38, S82-S88; Ruffolo, R. R., Jr., Boyle, D. A., Venuti, R. P. & Lukas, M. A. (1991) Drugs of Today, 27, 465-492; and Yue, T.-L., Cheng, H., Lysko, P. G., Mckenna, P. J., Feuerstein, R., Gu, J., Lysko, K. A., Davis, L. L. & Feuerstein, G. (1992) J. Pharmacol. Exp. Ther., 263, 92-98.

The most surprising observation from the current studies in which carvedilol is used to treat diabetic hypertensive patients is that said compound is able to maintain glycemic control. This result is surprising since there have been studies which report that beta-blocking agents, such as metoprolol, atenolol, and propranolol, significantly reduce glycemic control in hypertensive patients (Groop L, et al., Influence of beta-blocking drugs on glucose metabolism in patients with non-insulin-dependent diabetes mellitus. Acta Med Scand 1982; 211:7-12; Pollare et al., Sensitivity to insulin during treatment with atenolol and metoprolol: a randomised, double-blind study of the effects on carbohydrate and lipoprotein metabolism in hypertensive patients. Br Med J 1989;298:1152-7).

Pharmaceutical compositions of carvedilol may be administered to patients according to the present invention in any medically acceptable manner, preferably orally. For parenteral administration, the pharmaceutical composition will be in the form of a sterile injectable liquid stored in a suitable container such as an ampoule, or in the form of an aqueous or nonaqueous liquid suspension. The nature and composition of the pharmaceutical carrier, diluent or excipient will, of course, depend on the intended route of administration, for example whether by intravenous or intramuscular injection.

Pharmaceutical compositions of carvedilol for use according to the present invention may be formulated as solutions or lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as ethanol, polyvinyl-pyrrolidone, gelatin, hydroxy cellulose, acacia, polyethylene glycol, mannitol, sodium chloride or sodium citrate.

Alternatively, carvedilol may be encapsulated, tableted or prepared in a emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, ethanol, and water. Solid carriers include starch, lactose, calcium sulfate dihydrate, terra alba, magnesium stearate or stearic acid, talc, pectin, acacia, agar or gelatin. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

Dosing in humans for the treatment of disease according to the present invention should not exceed a dosage range of from about 6.25 to about 50 mg of carvedilol, preferably given twice daily. As one of ordinary skill in the art will readily comprehend, the patient should be started on a low dosage regimen of carvedilol, and monitered for well-known symptoms of intolerance, e.g., fainting, to such compound. Once the patient is found to tolerate such compound, the patient should be brought slowly and incrementally up to the maintenance dose. The preferred course of treatment is to start the patient on a dosage regimen of 6.25 mg, preferably given twice daily, for two weeks. The choice of initial dosage most appropriate for the particular patient is determined by the practitioner using well-known medical principles, including, but not limited to, body weight. In the event that the patient exhibits medically acceptable tolerance of the compound for two weeks, the dosage is doubled at the end of the two weeks and the patient is maintained at the new, higher dosage for two more weeks, and observed for signs of intolerance. This course is continued until the patient is brought to a maintenance dose. The preferred maintenance dose is 25 mg, preferably given twice daily, for patients having a body weight of up to 85 kg. For patients having a body weight of over 85 kg, the maintenance dose is between about 25 mg and about 50 mg, preferably given twice daily; preferably about 50 mg, preferably given twice daily.

### EXAMPLES

The primary objective of the study is to determine if the effect of carvedilol on glycemic control, as measured by the difference in the change from baseline in HbA1c, is superior to the effect of metoprolol in patients with controlled mild-to-moderate hypertension and non-insulin-dependent diabetes mellitus.

A total of 1210 patients at approximately 90-100 sites will be studied; 484 will be treated with carvedilol and 726 patients will be treated with metoprolol. Males or females 30 to 80 years of age with controlled hypertension accompanied by controlled non-insulin dependent diabetes mellitus will be enrolled.

Patients will have a documented history of mild to moderate hypertension (diastolic BP≧85 mmHg and ≦109 mmHg; systolic BP≧135 to ≦179 mmHg). All patients must be taking an angiotensin converting enzyme inhibitor or angiotensin receptor blocker either alone or as part of polypharmacy for control of their blood pressure. Patients who are prescribed angiotensin receptor blockers (ARBs) in place of angiotensin converting enzyme inhibitors are eligible for enrollment. A two week period for washout of all antihypertensive medications except angiotensin converting enzyme inhibitors (or ARBs) will precede randomization. Following the Washout Phase, blood pressure must be within the mild-to-moderate hypertensive range defined above for a patient to qualify for randomization.

At screening, patients must have had no changes to their antihypertensive regimen (medication or dose) during the month prior to screening and must have had no changes to their antidiabetic regimen (diet, medication including insulin, dose, or formulation) during the three months prior to screening. Patients taking oral antidiabetic medications must have an HbA1c of ≧6.5% and ≦8.5%. Patients managing their diabetes by diet alone must have an HbA1c of ≧6.5% and ≦7.5%. Patients must have a body mass index of 2245, and patients must be producing insulin as manifested by a fasting C-peptide of ≧0.6 ng/mL.

HbA1c levels will be determined at screening and then again following the two-week antihypertensive medication wash-out period, at a pre-randomization visit to be held 24 days prior to the scheduled randomization visit. Any patient whose HbA1c level has increased by greater than 0.5% between screening and the pre-randomization visit or whose fasting HbA1c level is not within the ranges stated above may have the HbA1c level determined once again within 3-5 days. Patients will be excluded if, upon re-testing, the HbA1c is out of the range defined for the appropriate therapy (diet or medication), or if the value at retest exceeds that obtained at screen by >0.5%.

In addition, patients will be excluded if: they have new onset hypertension (onset within the last month), or new onset diabetes mellitus (onset within three months). Patients will be excluded if they are taking beta-blockers for any indication, with the exception of eyedrops containing bets-blockers. Patients taking anorectic or any diet drugs, or practicing any dietary measures inconsistent with recommendations for patients with type II diabetes also must be excluded.

Patients also will be excluded for any of these cardiovascular conditions: uncontrollable or symptomatic arrhythmias; unstable angina, sick sinus syndrome, or second or third degree heart block (unless treated with a permanent, functioning pacemaker); symptomatic heart failure being actively treated, myocardial infarction or stroke within three months of screening; bradycardia heart rate <50 bpm). Patients will be excluded if they have a history of obstructive pulmonary disease requiring inhaled or oral bronchodilator therapy (e.g., asthma), clinically significant renal or liver disease, or active proliferative retinopathy. Patients with endocrine disorders (e.g., pheochromocytoma, active or untreated hypo- or hyperthyroidism) will be excluded, as will patients with any chronic disorder that requires chronic or intermittent use of corticosteroids, or continuous use of inhaled steroids.

Patients who are pregnant or who do not agree to use appropriate and adequate contraception for the duration of the study will be excluded. Patients with cancer or other systemic disease with reduced life expectancy (<12 months), and patients with a history of a psychological illness/condition that would interfere with their ability to understand or complete the requirements of the study also are ineligible for participation in the study.

Patients will be dropped from the study if diabetic control deteriorates such that insulin treatment is required, if their fasting blood glucose at any visit is >270 mg/dl (confirmed by immediate retesting), or if their blood pressure is not well-controlled within the target pressures identified.

This is a double-blind, randomized, parallel intent-to-treat trial. Patients will be randomized to either carvedilol or metoprolol after an antihypertensive medication washout period (2 weeks), and subsequent completion of baseline efficacy measurements. All antihypertensive medications except angiotensin converting enzyme (ACE) inhibitors [or angiotensin receptor blockers (ARBs)] are to be washed out during this interval. Patients whose hypertension is controlled solely by angiotension inhibition (ACE inhibitor or ARB) are eligible for entry into the study provided that they enter and complete the two week Washout period and their pre-randomization HbA1c levels fulfill the inclusion criteria. Baseline efficacy measurements are defined as the measurements obtained following completion of the washout period, but prior to receiving study medication. Patients will be stratified according to whether they are (or are not) taking angiotensin receptor blockers, and according to whether they are (or are not) taking thiazolidinediones (TZDs). Patients will be up-titrated during a phase of variable duration, to their target dose of study medication defined by a target blood pressure response (see below), and will enter the maintenance phase at this dose.

Patients randomized to carvedilol will begin up-titration at 12.5 mg total daily dose (6.25 mg bid; dose level 1) for 1 week. If blood pressure is not reduced (diastolic to ≦80 mmHg or systolic to ≦130 mmHg), the dose will be increased to dose level 2 (25 mg total daily dose, 12.5 mg bid) for 1 week. If the patient does not achieve the target blood pressure, the dose will be increased to dose level 3 (50 mg total daily dose, 25.0 mg bid), the highest study medication dose.

Patients randomized to metoprolol tartrate will begin up-titration at 100 mg total daily dose (50 mg bid; dose level 1) for 1 week. If blood pressure is not reduced (diastolic to ≦80 mmHg or systolic to ≦130 mmHg), the dose will be increased to dose level 2 (200 mg total daily dose, 100 mg bid) for 1 week. If the patient does not achieve the target blood pressure, the dose will be increased to dose level 3 (400 mg total daily dose, 200 mg bid), the highest study medication dose.

It is permissable, if medically necessary or preferred by the investigator, to titrate any given patient at two week intervals. This may include routine two-week intervals at each step of titration, or use of a two-week interval at any individual titration step.

If the hypertension is not adequately controlled by either carvedilol or metoprolol, hydrochlorothiazide (up to 12.5 mg) will be added. If necessary to further reduce blood pressure, a dihydropyridine calcium channel antagonist may be added. The dose of angiotension converting enzyme inhibitor (or ARB) and antidiabetic medications including insulin must remain stable throughout the study. Provisions are provided in the detailed protocol for management of those patients who lose glycemic control or whose blood pressure goes out of control. If medically appropriate, dosage levels of all other concomitant medications should remain unchanged while the patient is enrolled in the study.

The study includes six phases:

Screening/Washout Phase (14 days)—The patient is evaluated to establish eligibility for randomization. When all study criteria are satisfied (including all laboratory results) and 2 weeks has elapsed since the HbA1c at screening has been drawn, the patient is instructed to suspend present antihypertensive therapy [except ACE inhibition or ARB] for the washout period of 2 weeks. Patients who are not on polypharmacy, and are only on an ACE inhibitor or ARB are still required to complete this Washout period to ascertain that their fasting HbA1c meets the randomization criteria. A patient who fails the first screening may be reassessed for the study one additional time.

Baseline/Randomization Phase—The Screening/Washout Phase ends with baseline assessments and randomization. The patient must return for a “pre-randomization” visit 24 days prior to the end of the washout period for determination of fasting plasma HbA1c. An increase of >0.5% in HbA1c relative to the screening visit (confirmed by re-measurement) would make a patient ineligible for randomization. At the end of the 14 day washout period, when the “pre-randomization” HbA1c value is in hand (and acceptable), and if the patient's blood pressure is within the specified ranges, the patient should be randomized. The first dose of study medication at Dose Level 1 will be administered the same day.

Titration Phase (2-11 weeks)—The Titration Phase begins with the first dose of study medication. After the first dose of study medication at Dose Level 1, the patient returns to the clinic in one week for evaluation and advancement to the next level of study medication (if necessary) to achieve the target blood pressure.

The aim of the Titration Phase is to achieve the target blood pressure response before entry of the patient into the Maintenance Phase. The blood pressure response criteria differ based on the entry criteria at the baseline/randomization visit. If the baseline diastolic BP was ≧85 mmHg, the response is defined as ≦80 mmHg. If the baseline diastolic BP was less than 85 mmHg and the systolic BP was ≧135 mmHg, the response is defined as systolic BP ≦130 mmHg. For patients with both elevated systolic and diastolic pressures, achieving the target diastolic blood pressure is sufficient in cases where it is proving difficult to reduce systolic blood pressure to ≦130 mmHg.

The Titration Phase is variable in length. If necessary to add hydrochlorothiazide (up to 12.5 mg daily) and then a dihydropyridine calcium channel blocker to obtain the target blood pressure response, titration will last a minimum of 6 weeks.

Once a patient has achieved that target blood pressure, and maintained that pressure for one week (i.e, two consecutive visits), he is eligible for entry into the maintenance phase.

Maintenance Phase (5 months)—The patient continues on the dose sufficient during Titration to attain the target blood pressure, and returns to the clinic after 1, 2, 3, 4, and 5 months of maintenance therapy.

Down-Titration Phase (≦2 weeks)—At the end of five months of maintenance therapy, the patient will be down-titrated by reducing the study medication total daily dosage incrementally (by dose level) with one-week intervals between each dose level. The time to complete this phase is variable and dependent on the Maintenance Phase drug regimen (e.g., drug level). The patient returns for an End of Down-Titration visit 1-3 days after taking the last dose of Level 1 study medication. The patient then is scheduled for a Follow-up visit 2 weeks after the End of Down-Titration visit. Patients who were prescribed hydrochlorothiazide and a calcium channel blocker to achieve the target blood pressure will have only the blinded study medication removed by down-titration, leaving the patient still taking the diuretic and CCB during the two week follow-up phase. Management of the patient from that point forward is at the discretion of the investigator.

Follow-up—The patient returns two weeks after the last dose of blinded study medication for the final safety assessments. The patient completes the study with this visit.

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

1. A method of maintaining glycemic control in diabetic hypertensive patients which comprises administering carvedilol to a subject in need thereof.

2. The method according to claim 1 which comprises administering carvedilol in a dosage range from about 6.25 to about 25 mg given twice daily.

3. The method according to claim 1 which comprises administering carvedilol in a maintenance dose of about 25 mg given twice daily.

4-6. (Cancelled)
